(19) United States
(12) Patent Application Publication
(10) Pub. No.: US 2026/0301853 A1
(43) Pub. Date: Oct. 1, 2026

(54) PHYLOGENETIC INFERENCE CARRIED OUT CONCURRENTLY WITH A SEQUENCING OPERATION

(71) Applicant: International Business Machines Corporation, Armonk, NY (US)

(72) Inventors: RITESH VIJAY KRISHNA, Sale (GB); LAURA-JAYNE GARDINER, Wirral (GB); VADIM ELISSEEV, Warrington (GB)

(21) Appl. No.: 19/564,577

(22) Filed: Mar. 12, 2026

(30) Foreign Application Priority Data
Apr. 1, 2025 (GB) 2504846.3

(51) Int. Cl. G16B 10/00; G16B 30/10; G16B 50/30

(52) U.S. Cl. CPC G16B 10/00; G16B 30/10; G16B 50/30

(57) ABSTRACT

A method, computer program product, and computer system infer phylogenic relationships concurrently with a sequencing operation. The inferring includes, while sequencing data is in production by a sequencing machine, accessing the sequencing data, mapping sequence reads from the accessed sequencing data to identify sequence reads corresponding to orthologous genes of interest, and placing the identified sequence reads into respective namespaces for the corresponding orthologous genes in the in-memory database.

### BACKGROUND

The present invention relates to phylogenetic inference, and more specifically, to phylogenetic inference carried out concurrently with a sequencing operation.

Phylogenetic analysis is a component of comparative genomics and can be used to identify new genetic variants and study differences between existing ones. Phylogenetic reconstruction techniques can be distance-based or character-based. These techniques are based on aligned sequence datasets and follow a linear set of processes to infer a tree structure. Some phylogenic analysis methods use a computational method to infer a phylogenetic tree using deep learning and clustering methods. These methods use a traditional compute environment on a dataset available after the sequencing process is finished.

### SUMMARY

In some aspects, the techniques described herein relate to a computer-implemented method for generating phylogenetic inferences using a distributed computing environment with an in-memory database, the computer-implemented method including: inferring phylogenic relationships concurrently with a sequencing operation, wherein the inferring includes, while sequencing data is in production by a sequencing machine: accessing the sequencing data; mapping sequence reads from the accessed sequencing data to identify sequence reads corresponding to orthologous genes of interest; and placing the identified sequence reads into respective namespaces for the corresponding orthologous genes in the in-memory database.

Further embodiments are directed to a system, which includes a memory and a processor communicatively coupled to the memory, wherein the processor is configured to perform the method. Additional embodiments are directed to a computer program product, which includes a computer readable storage medium having program instructions embodied therewith, the program instructions executable by a processor to cause a device to perform the method.

The above summary is not intended to describe each illustrated embodiment or every implementation of the present disclosure.

### BRIEF DESCRIPTION OF THE DRAWINGS

Embodiments of the present invention will now be described, by way of example only, with reference to the accompanying drawings:

FIG. 1 is a block diagram illustrating an example of a distributed computing environment, according to some embodiments.

FIG. 2 is a swim-lane flow diagram illustrating processes carried out at each of the insertion stage, the local tree inference stage, and the global tree inference stage, according to some embodiments.

FIG. 3A is a flow diagram illustrating an example process that can take place at the insertion stage, according to some embodiments.

FIG. 3B is a flow diagram illustrating an example process that can take place at the local tree inference stage, according to some embodiments.

FIG. 4A is a schematic diagram showing the sequencing machine running concurrently with the insertion stage of FIG. 2 to generate namespaces in an in-memory database, according to some embodiments.

FIG. 4B is a schematic diagram illustrating trees being generated for the individual namespaces while a concurrent insertion stage is taking place, according to some embodiments.

FIG. 5 is a block diagram illustrating a computing environment, according to some embodiments.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numbers may be repeated among the figures to indicate corresponding or analogous features.

### DETAILED DESCRIPTION

Embodiments of a method, system, and computer program product are provided for phylogenetic inference that may be applied concurrently with a sequencing operation. This may result in near real-time phylogenetic inference during the sequencing operation.

Clause 1. A computer-implemented method for phylogenetic inference carried out concurrently with a sequencing operation using a distributed computing environment with an in-memory database, said method comprising: accessing sequencing data whilst in production by a sequencing machine; mapping sequence reads to identify sequence reads belonging to orthologous genes of interest; and placing an identified sequence read into an in-memory database namespace for the orthologous genes to provide virtual groupings of sequence reads from a same gene enabling phylogenetic relationship inference while sequencing is in operation.

The described method has the advantage of performing phylogenetic analysis, faster and in near real-time alongside a sequencing operation.

Clause 2. The method of clause 1, including: at first intervals for each database namespace, gathering the sequence reads to create a representation format and provide phylogenetic inference trees for the namespace.

Clause 3. The method of clause 2, including: at second intervals, gathering all phylogenetic inference trees from the namespaces and apply computational approaches to infer a global tree that takes into account all the orthologous genes.

Clause 4. The method of clause 3, wherein the placing an identified sequence read into an in-memory database namespace, the gathering of sequence reads at first intervals to create a representation format and provide phylogenetic inference trees for the namespace, and the gathering of all phylogenetic inference trees from the namespaces at second intervals and applying computational approaches to infer a global tree, take place concurrently with the first intervals being more frequent than the second intervals.

Clause 5. The method of any one of the preceding clauses, including providing a feedback signal to halt the sequencing process when relevant phylogenetics information is available to the user.

Clause 6. The method of any one of the preceding clauses including providing visualization of the inferred phylogenetic trees while genome sequencing is in operation.

Clause 7. The method of any one of the preceding clauses, wherein the mapping sequence reads uses short read alignment to identify if a sequence read belongs to the orthologous genes of interest.

Clause 8. The method of any one of the preceding clauses, including placing non-identified sequence reads into the in-memory database without namespacing.

Clause 9. The method of any one of clauses 2 to 8, wherein gathering sequence reads to create a representation format for phylogenetic inference uses a distributed multiple sequence alignment to create the representation format.

Clause 10. The method of clause 9, including passing the representation format to a distributed phylogenetic inference program for providing phylogenetic inference trees for the namespace.

Clause 11. The method of clause 9 or 10, including placing the representation format back into the database namespace to reduce cost as new sequences are allocated to the database namespace.

Clause 12. A system for phylogenetic inference carried out concurrently with a sequencing operation using a distributed computing environment with an in-memory database, said method comprising: a phylogenetic interference system including: a data accessing component for accessing sequencing data whilst in production by a sequencing machine; a mapping component for mapping sequence reads to identify sequence reads belonging to orthologous genes of interest; and a placing component for placing an identified sequence read into an in-memory database namespace for the orthologous genes to provide virtual groupings of sequence reads from a same gene enabling phylogenetic relationship inference while sequencing is in operation.

Clause13. The system of clause 12, including: a local tree inference component for, at first intervals for each database namespace, gathering the sequence reads to create a representation format and provide phylogenetic inference trees for the namespace.

Clause 14. The system of clause 13, including: a global tree inference component for, at second intervals, gathering all phylogenetic inference trees from the namespaces and apply computational approaches to infer a global tree that takes into account all the orthologous genes.

Clause15. The system of any one of clauses 12 to 14, including a feedback component for providing a feedback signal to halt the sequencing process when relevant phylogenetics information is available to the user.

Clause 16. The system of any one of clauses 12 to 15, including a visualization component for providing a visualization of the inferred phylogenetic trees while genome sequencing is in operation.

Clause 17. The system of any one of clauses 12 to 16, wherein the local tree inference component uses a distributed multiple sequence alignment (MSA) program to create the representation format.

Clause 18. The system of any one of clauses 12 to 17, wherein the local tree inference component is for passing the representation format to a distributed phylogenetic inference program for providing phylogenetic inference trees for the namespace.

Clause 19. The system of any one of clauses 12 to 18, wherein the local tree inference component is for placing the representation format back into the database namespace to reduce cost as new sequences are allocated to the database namespace.

Clause20. A computer program stored on a computer readable medium and loadable into the internal memory of a digital computer, comprising software code portions, when said program is run on a computer, for performing the method steps of any of the clauses 1 to 11.

Phylogenetics is a cornerstone of comparative genomics. Various subfields in biology, like pathogen genomics, viral genomics, metagenomics etc., depend on phylogenetic analysis to identify new genetic variants and to understand evolution and differences between species. Phylogenetic reconstruction techniques are either distance-based or character-based and are computationally expensive. These techniques are based on aligned sequence datasets and follow a linear set of processes to infer a tree structure. Given the rise in genome sequencing and considerable compute time associated with tree inference, it is important to be able to infer the phylogenetic structure for a given dataset in reduced time.

To highlight this, an exemplary application for phylogenetic analysis is to compare a new viral strain with pre-existing strains based on genetic sequence, for example, as has been vital in the analysis of COVID-19. Typically, the investigation of the organism of interest (in this example a virus) is derived from genome sequencing or omic technologies, for example, of patient samples who are likely to be infected. There is a clear need for phylogenetic analysis to be as streamlined as possible in cases such as this where a fast response to findings is required.

Known methods use a computational method to infer a phylogenetic tree using deep learning and clustering methods. These methods use a traditional compute environment on a dataset available after the sequencing process is finished.

Embodiments of the present disclosure provide a phylogenetic inference system in the form of a software toolkit that may include a suite of computational methods to infer phylogenetic relationships at the level of predetermined orthologous genes of interest. An orthologous gene is a gene in different species that evolved from a common ancestor by speciation.

Relationships may be inferred both at the local or individual gene level, as well as the global level, using sets of all orthologous genes of interest. The method is disclosed in context of real-time sequencing, i.e., at the time while the sequencing operation is taking place, eventually aiming for inference of phylogenetic relationship in near-real time. The software toolkit may include a computational method to allow visualization of the inferred phylogenetic trees while genome sequencing is in operation. The software toolkit may be provided on a distributed system to exploit the speed and efficiency of distributed in-memory databases.

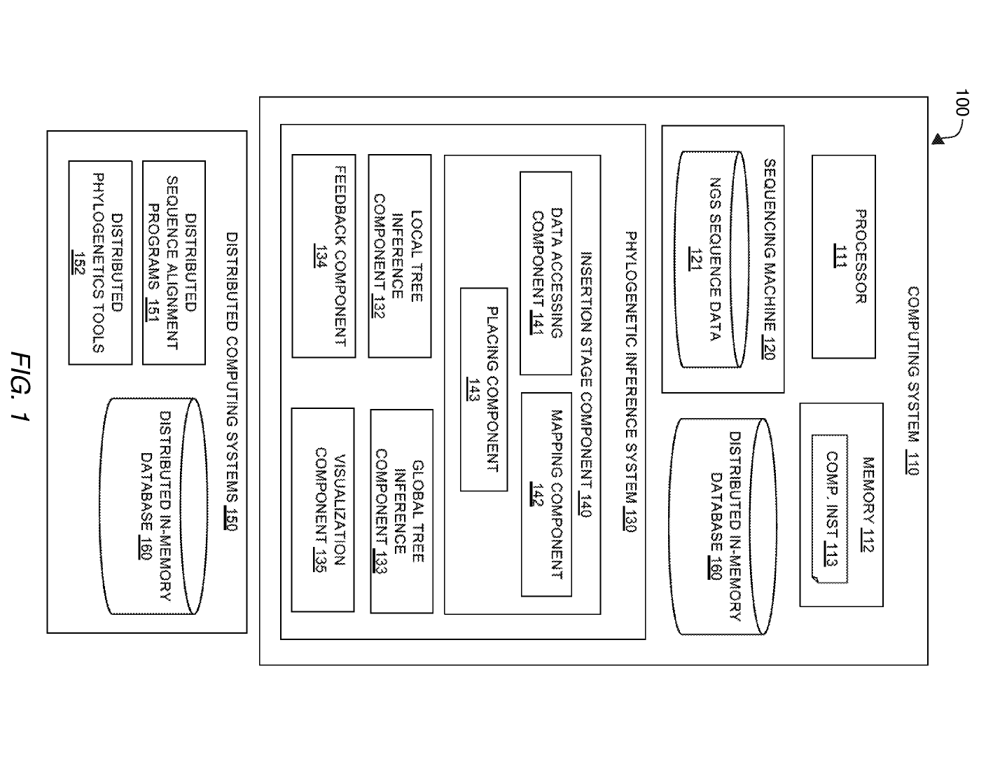

FIG. 1 is a block diagram illustrating an example of a distributed computing environment 100, according to some embodiments. The distributed computing environment 100 includes a computing system 110 at which a sequencing machine 120 may operate and at which a phylogenetic inference system 130 as described herein may be provided. The computing system 110 can interact with other distributed computing systems 150 of the distributed computing environment 100 to use distributed in-memory databases 160.

Each of the distributed computing systems 110, 150 may include at least one processor 111, a hardware module, or a circuit for executing the functions of the described components which may be software units executing on the at least one processor. Multiple processors running parallel processing threads may be provided enabling parallel processing of some or all of the functions of the components. Memory 112 may be configured to provide computer instructions 113 to the at least one processor 111 to carry out the functionality of the components.

The described phylogenetic inference system 130 is provided in the distributed computing environment 100 with an in-memory database 160 as in-memory databases 160 rely primarily on memory for data storage to enable minimal response times by eliminating the need to access disks.

The phylogenetic inference system 130 may be linked with the computing system 110 connected to a genome sequencing machine 120, so that the phylogenetic inference system 130 can tap the sequence data 121 whilst in production. The data can then be streamed to the distributed computing systems 150 which may be located close to the sequencing machine 120 or in cloud via high bandwidth connectivity. The distributed computing environment 100 hosts a distributed in-memory database 160 operating on key-value operations. The computing environment 100 also hosts distributed community usable bioinformatics tools. The community usable bioinformatics tools may include distributed versions of multiple sequence alignment (MSA) programs 151 and distributed phylogenetics program 152.

The phylogenetic interference system 130 includes an insertion stage component 140 including a data accessing component 141 for accessing sequencing data 121 while in production by a sequencing machine 120. The insertion stage component 140 also includes a mapping component 142 for mapping sequence reads to identify sequence reads belonging to orthologous genes of interest and a placing component 143 for placing an identified sequence read into a namespace for the orthologous genes in an in-memory database 160 to provide virtual groupings of sequence reads from a same gene enabling phylogenetic relationship inference while sequencing is in operation. The placing component 143 may also be for placing non-identified sequence reads into the in-memory database 160 without namespacing. Database namespaces provide a virtual compartmentalization of the database address range to create individual logical spaces.

The phylogenetic interference system 130 can include a local tree inference component 132 for, at first intervals for each database namespace, gathering the sequence reads to create a representation format and provide phylogenetic inference trees for the namespace. The local tree inference component 132 may use a distributed MSA program 151 to create the representation format. The local tree inference component 132 may also pass the representation format to a distributed phylogenetic inference program 152 for providing phylogenetic inference trees for the namespace. The local tree inference component 132 may place the representation format back into the database namespace to reduce cost as new sequences are allocated to the database namespace.

The phylogenetic interference system 130 includes a global tree inference component 133 for, at second intervals, gathering all phylogenetic inference trees from the namespaces and apply computational approaches to infer a global tree that takes into account all the orthologous genes.

The phylogenetic interference system 130 may include a feedback component 134 for providing a feedback signal to halt the sequencing process when relevant phylogenetics information is available to the user. The feedback may be provided in the form of interrupt event or signal generated by the feedback component 134. This may be generated in response to an input received from a user. When the program sees the interrupt code, it conveys that message to the sequencer to halt.

The phylogenetic interference system 130 may include a visualization component 135 for providing visualization of the inferred phylogenetic trees while genome sequencing is in operation. The visualization may display the inferred phylogenetic tree using existing tree building software. The displayed trees may be interactive in nature and may be visualized in a browser or an application.

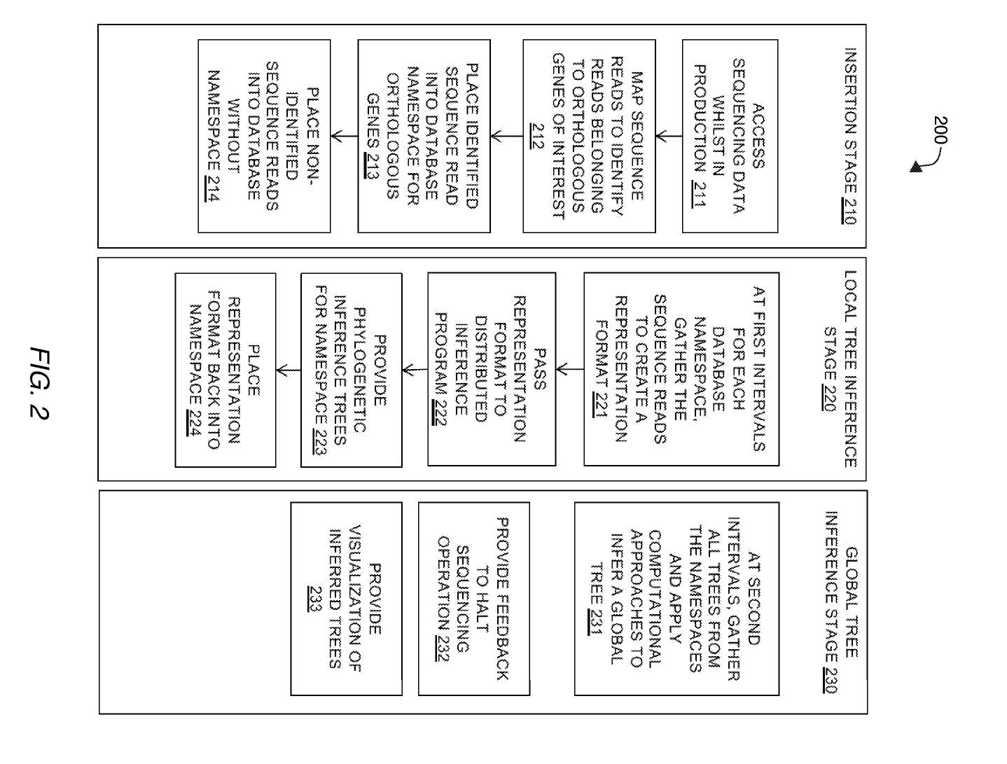

FIG. 2 is a swim-lane flow diagram illustrating processes 200 carried out at each of the insertion stage 210, the local tree inference stage 220, and the global tree inference stage 230, according to some embodiments. Process 200 can include inferring phylogenetic relationships between species for each set of predetermined orthologous genes and between species for a global set of orthologous genes while genome sequencing is in operation. Process 200 may optionally be carried out using components of distributed computing environment 100 (FIG. 1).

As shown at operation 211, the insertion stage 210 can include accessing sequencing data while in production by a sequencing machine and, as shown at operation 212, mapping sequence reads to identify sequence reads belonging to orthologous genes of interest. Mapping the sequence reads at operation 212 may include using short read alignment to identify if a sequence read belongs to the orthologous genes of interest. As shown at operation 213, the insertion stage 210 may include placing an identified sequence read into an in-memory database namespace for the orthologous genes to provide virtual groupings of sequence reads from a same gene. This can enable phylogenetic relationship inference while sequencing is in operation. As shown at operation 214, the insertion stage 210 may also include placing non-identified sequence reads in the in-memory database without namespacing.

The local tree inference stage 220 can include inferring phylogenetic relationships between species for each database namespace set of orthologous genes while sequencing is in operation. As shown at operation 221, at first intervals for each database namespace, the local tree inference stage 220 may include gathering the sequence reads to create a representation format. Gathering the sequence reads to create a representation format for phylogenetic inference at operation 221 may include using a distributed multiple sequence alignment (MSA) to create the representation format.

As shown at operation 222, the local tree inference stage 220 may include passing the representation format to a distributed phylogenetic inference program (e.g., distributed phylogenetic inference program 152) that, as shown at operation 223, can provide phylogenetic inference trees for the namespace. As shown at operation 224, the representation format may be placed back into the database namespace to reduce cost as new sequences are allocated to the database namespace.

The global tree inference stage 230 can include inferring phylogenetic relationships between species for a global set of orthologous genes while genome sequencing is in operation. As shown at block 231, at second intervals, the global tree inference stage 230 may include gathering all phylogenetic inference trees from the namespaces and applying computational approaches to infer a global tree that takes into account all the orthologous genes.

The insertion stage 210, the local tree inference stage 220, and the global tree inference stage 230 may take place concurrently, where the inference operations continually operate, and where the first intervals are more frequent than the second intervals.

As shown at operation 232, the global tree inference stage 230 can also include providing a feedback signal to halt the sequencing process when relevant phylogenetics information is available to the user. Further, as shown at operation 233, the global tree inference stage 230 can include providing a visualization of the inferred phylogenetic trees while genome sequencing is in progress.

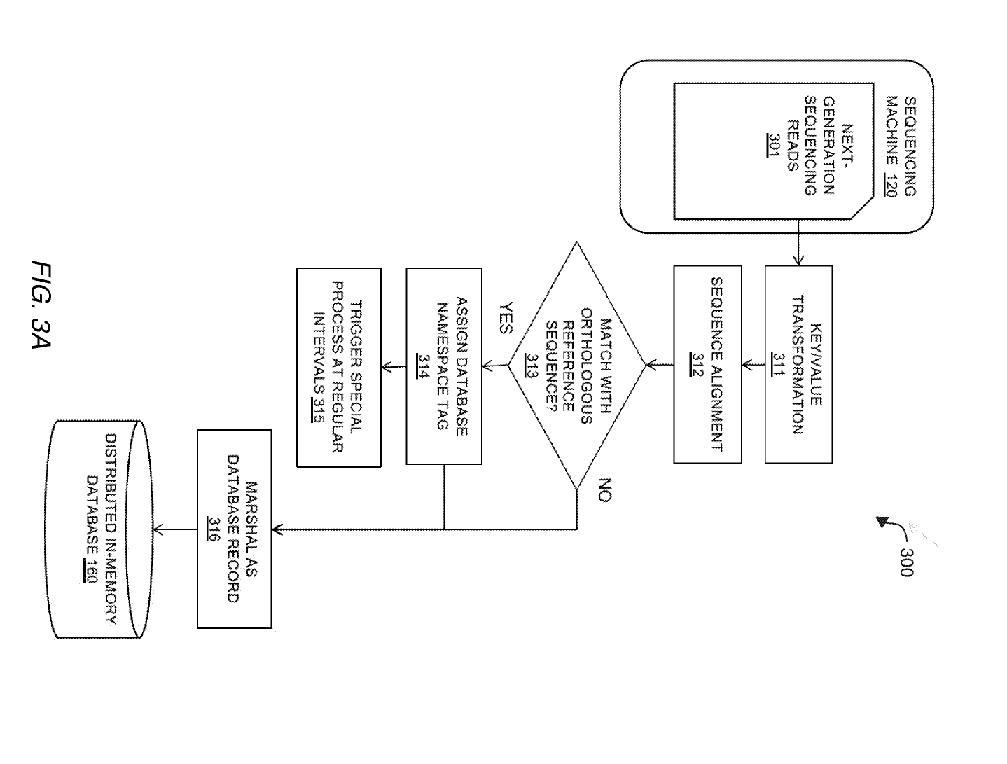

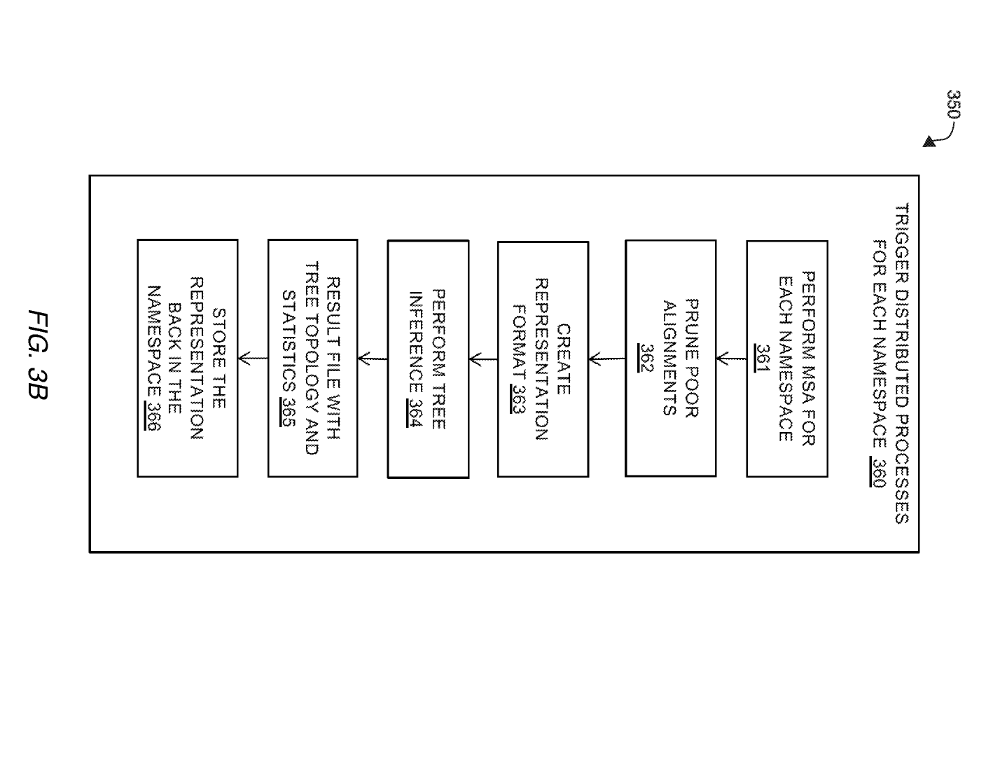

FIG. 3A is a flow diagram illustrating an example process 300 that can take place at the insertion stage 210, according to some embodiments. As shown at operation 301, genome sequencing machine 120 can write next-generation sequence (NGS) reads on disk, for example, at a secondary storage device, as soon as the sequence is read. Each read has an identifier and, as shown at operation 311, a data-structure can be created with a key/value pair to represent the sequence information, where the identifier is used as the key and the sequence itself is used as the value. This operation continues as long as sequencing is taking place. The phylogenetic inference system 130 can read data from the sequencer’s secondary storage device as soon as any new data is produced.

The key-value pairs can be transformed at operation 311. Sequence alignment can be carried out at operation 312 by mapping the sequence read using a short read alignment tool, for example, a Burrow-Wheeler Aligner (BWA), to identify if the read belongs to the orthologous genes of interest.

It can be determined whether there is a match with an orthologous reference sequence at operation 313. If the read belongs to any orthologous gene (313: yes), then the read can be placed into the corresponding database namespace of distributed in-memory database 160 by being assigned a database namespace tag (operation 314). This can allow for virtual grouping of sequence reads from the same gene. As shown at operation 315, the local tree inference stage 220 can be triggered at intervals for each namespace. If no match is found (313: no), the read can be marshalled as a data base record (operation 316), and a simple insertion in the database 160 without any special namespacing can be done.

FIG. 3B is a flow diagram illustrating an example process 350 that can take place at the local tree inference stage 220, according to some embodiments. At intervals that may be determined by criteria of choice, such as an increase in the namespace size, the local tree inference stage process can be triggered (operation 360), which in turn can start individual child operations 361–366 to work with each existing namespace.

In each local tree inference stage process 360, the sequence reads are gathered from the process’ namespace. Each local tree inference stage process 360 includes performing distributed MSA (operation 361) and pruning for poor alignments using alignment pruning software (operation 362) to create a representation format in the form of a community-accepted textual file format to represent phylogenetic relationships (operation 363).

The representation format is then passed on to a distributed phylogenetic inference program to infer a tree structure for reads in that namespace (operation 364). This creates a resultant file (operation 365) with a tree topology and statistics for the corresponding orthologous gene. The representation is stored (operation 366) back in the namespace and may be used for reducing the cost of MSA as new sequences get allocated to the namespace.

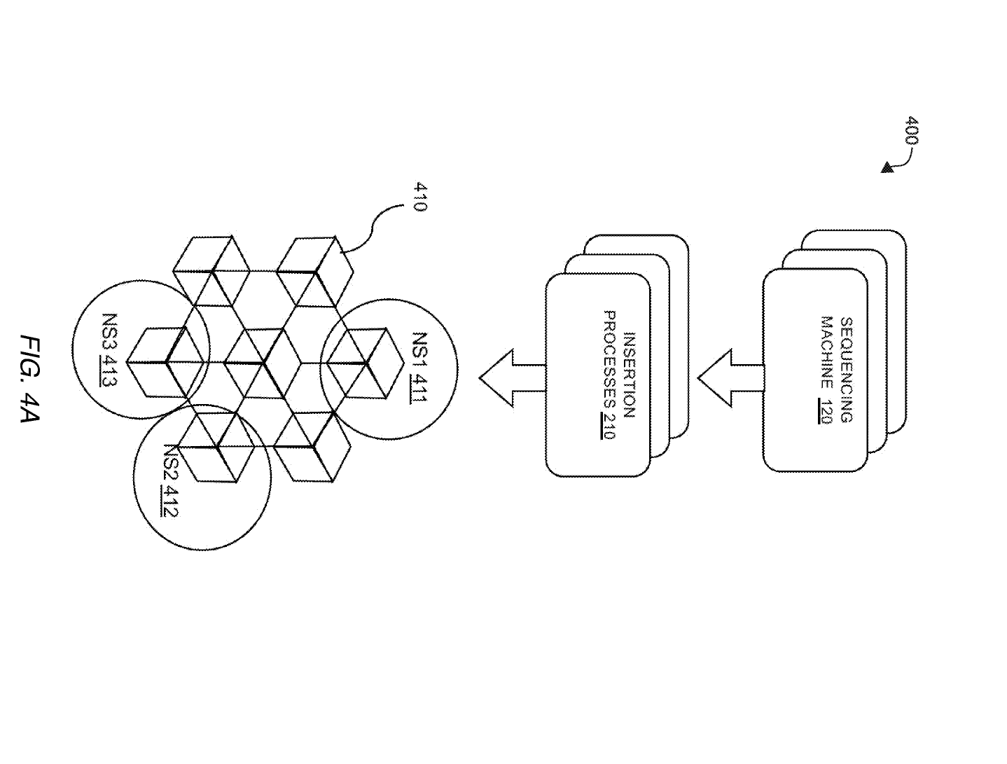

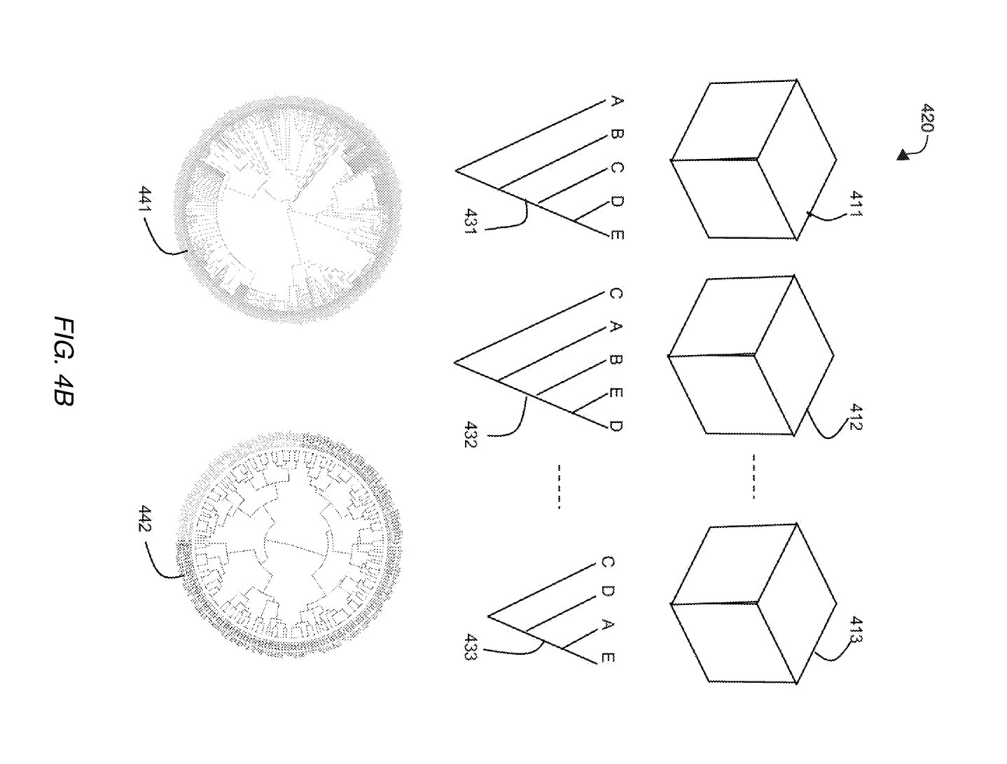

FIG. 4A is a schematic diagram 400 showing the sequencing machine 120 running concurrently with the insertion stage 210 of FIG. 2 to generate namespaces 411–413 in an in-memory database 410, according to some embodiments. In-memory database 410 may be substantially the same as the in-memory database 160 illustrated in FIG. 1. FIG. 4B is a schematic diagram 420 illustrating trees 431–433 being generated for the individual namespaces 411–413 while the concurrent insertion stage 210 (shown in FIGS. 2 and 4A) is taking place. At regular intervals, determined by the criteria of choice, a global tree inference stage 230 (shown in FIG. 2) can be triggered. The global tree inference stage 230 can include gathering all of the trees 431–433 from all of the namespaces 411–413 in the in-memory database 410 and applying supermatrix 441 and/or supertree 442 phylogeny computational approaches to infer a global tree 441, 442 that takes all the orthologous genes into consideration.

All three stages 210, 220, and 230 can operate concurrently, with the insertion stage 210 being the most frequent, local tree inference stage 220 relatively less frequent, and the global tree inference stage 230 being the least frequent. The in-memory database 160 can ensure grouping of sequence reads according to their orthologous relationships and continuous accessibility of data while the data is being collected during the sequencing. The fast and lean nature of in-memory database 160 can allow for quick insertion and exploitation of data as there is no need to perform secondary I/O operations between the stages of mapping, MSA, and tree inference as required in the traditional setting. All operations and data storage between the stages 210, 220, and 230 can be performed in-memory using in-memory database technology. The distributed MSA and tree inference (including global tree inference) can use the data from each namespace at regular intervals to infer and update trees at regular intervals. The phylogenetic inference system 130 software toolkit may also provide a visualization plugin (e.g., visualization component 135) that can display updated trees from different namespaces, as well as the global trees inferred in the global tree inference stage 230.

Using, e.g., feedback component 134, phylogenetic inference system 130 may generate a user-friendly feedback signal to halt the ongoing sequencing process as soon as relevant phylogenetics information is available to the user. The phylogenetic inference system 130 software toolkit can exploit in-memory, distributed databases to enable near real-time phylogenetic analysis with the built-in capability to halt the ongoing sequencing process as soon as the aims for doing phylogenetic analysis have been met.

In some embodiments, the above-described method provides ways to infer phylogenetics trees on the sequencing data in production (real-time data generation) and uses a compute architecture based on distributed in-memory technologies, provding fresh conceptual gains, functionalities, data representation and flow, speed-ups, and novel ways to utilize a group of technologies.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits / lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

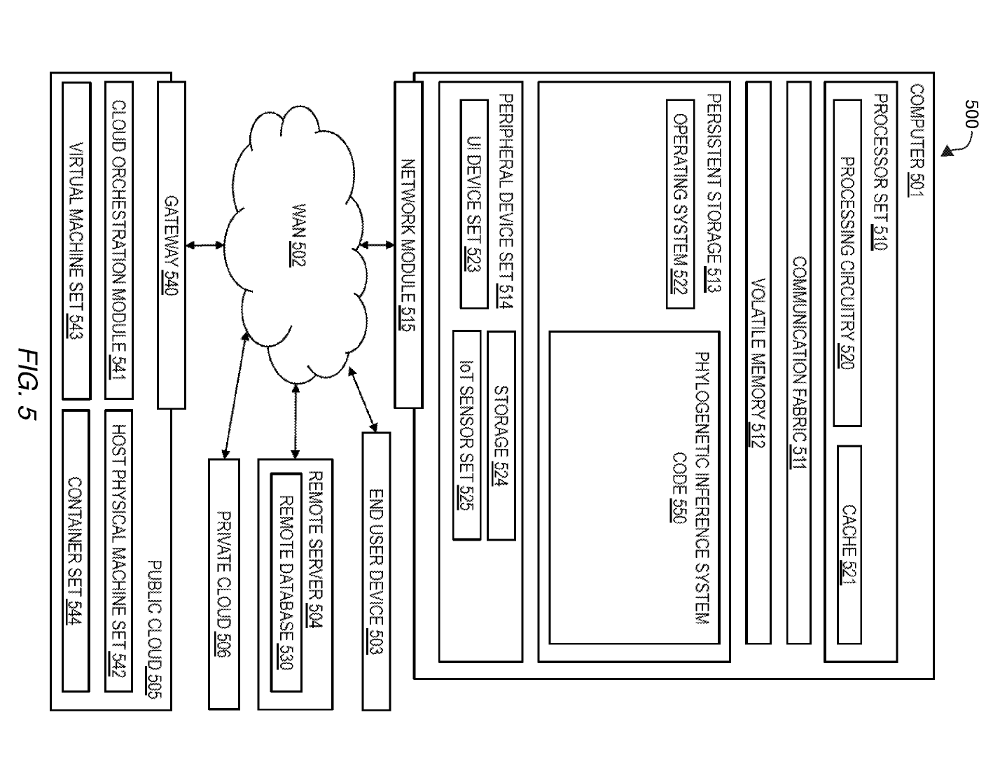

FIG. 5 is a block diagram illustrating a computing environment 500, according to some embodiments. Computing environment 500 contains an example of an environment for the execution of at least some of the computer code involved in performing the inventive methods, such as phylogenetic inference system code 550. In addition to block 550, computing environment 500 includes, for example, computer 501, wide area network (WAN) 502, end user device (EUD) 503, remote server 504, public cloud 505, and private cloud 506. In this embodiment, computer 501 includes processor set 510 (including processing circuitry 520 and cache 521), communication fabric 511, volatile memory 512, persistent storage 513 (including operating system 522 and block 550, as identified above), peripheral device set 514 (including user interface (UI) device set 523, storage 524, and Internet of Things (IoT) sensor set 525), and network module 515. Remote server 504 includes remote database 530. Public cloud 505 includes gateway 540, cloud orchestration module 541, host physical machine set 542, virtual machine set 543, and container set 544.

COMPUTER 501 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 530. As is well understood in the art of computer technology, and depending upon the technology, performance of a computer-implemented method may be distributed among multiple computers and/or between multiple locations. On the other hand, in this presentation of computing environment 500, detailed discussion is focused on a single computer, specifically computer 501, to keep the presentation as simple as possible. Computer 501 may be located in a cloud, even though it is not shown in a cloud in FIG. 5. On the other hand, computer 501 is not required to be in a cloud except to any extent as may be affirmatively indicated.

PROCESSOR SET 510 includes one, or more, computer processors of any type now known or to be developed in the future. Processing circuitry 520 may be distributed over multiple packages, for example, multiple, coordinated integrated circuit chips. Processing circuitry 520 may implement multiple processor threads and/or multiple processor cores. Cache 521 is memory that is located in the processor chip package(s) and is typically used for data or code that should be available for rapid access by the threads or cores running on processor set 510. Cache memories are typically organized into multiple levels depending upon relative proximity to the processing circuitry. Alternatively, some, or all, of the cache for the processor set may be located “off chip.” In some computing environments, processor set 510 may be designed for working with qubits and performing quantum computing.

Computer readable program instructions are typically loaded onto computer 501 to cause a series of operational steps to be performed by processor set 510 of computer 501 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 521 and the other storage media discussed below. The program instructions, and associated data, are accessed by processor set 510 to control and direct performance of the inventive methods. In computing environment 500, at least some of the instructions for performing the inventive methods may be stored in block 550 in persistent storage 513.

COMMUNICATION FABRIC 511 is the signal conduction path that allows the various components of computer 501 to communicate with each other. Typically, this fabric is made of switches and electrically conductive paths, such as the switches and electrically conductive paths that make up busses, bridges, physical input / output ports and the like. Other types of signal communication paths may be used, such as fiber optic communication paths and/or wireless communication paths.

VOLATILE MEMORY 512 is any type of volatile memory now known or to be developed in the future. Examples include dynamic type random access memory (RAM) or static type RAM. Typically, volatile memory 512 is characterized by random access, but this is not required unless affirmatively indicated. In computer 501, the volatile memory 512 is located in a single package and is internal to computer 501, but, alternatively or additionally, the volatile memory may be distributed over multiple packages and/or located externally with respect to computer 501.

PERSISTENT STORAGE 513 is any form of non-volatile storage for computers that is now known or to be developed in the future. The non-volatility of this storage means that the stored data is maintained regardless of whether power is being supplied to computer 501 and/or directly to persistent storage 513. Persistent storage 513 may be a read only memory (ROM), but typically at least a portion of the persistent storage allows writing of data, deletion of data and re-writing of data. Some familiar forms of persistent storage include magnetic disks and solid state storage devices. Operating system 522 may take several forms, such as various known proprietary operating systems or open source Portable Operating System Interface-type operating systems that employ a kernel. The code included in block 550 typically includes at least some of the computer code involved in performing the inventive methods.

PERIPHERAL DEVICE SET 514 includes the set of peripheral devices of computer 501. Data communication connections between the peripheral devices and the other components of computer 501 may be implemented in various ways, such as Bluetooth connections, Near-Field Communication (NFC) connections, connections made by cables (such as universal serial bus (USB) type cables), insertion-type connections (for example, secure digital (SD) card), connections made through local area communication networks and even connections made through wide area networks such as the internet. In various embodiments, UI device set 523 may include components such as a display screen, speaker, microphone, wearable devices (such as goggles and smart watches), keyboard, mouse, printer, touchpad, game controllers, and haptic devices. Storage 524 is external storage, such as an external hard drive, or insertable storage, such as an SD card. Storage 524 may be persistent and/or volatile. In some embodiments, storage 524 may take the form of a quantum computing storage device for storing data in the form of qubits. In embodiments where computer 501 is required to have a large amount of storage (for example, where computer 501 locally stores and manages a large database) then this storage may be provided by peripheral storage devices designed for storing very large amounts of data, such as a storage area network (SAN) that is shared by multiple, geographically distributed computers. IoT sensor set 525 is made up of sensors that can be used in Internet of Things applications. For example, one sensor may be a thermometer and another sensor may be a motion detector.

NETWORK MODULE 515 is the collection of computer software, hardware, and firmware that allows computer 501 to communicate with other computers through WAN 502. Network module 515 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 515 are performed on the same physical hardware device. In other embodiments (for example, embodiments that utilize software-defined networking (SDN)), the control functions and the forwarding functions of network module 515 are performed on physically separate devices, such that the control functions manage several different network hardware devices. Computer readable program instructions for performing the inventive methods can typically be downloaded to computer 501 from an external computer or external storage device through a network adapter card or network interface included in network module 515.

WAN 502 is any wide area network (for example, the internet) capable of communicating computer data over non-local distances by any technology for communicating computer data, now known or to be developed in the future. In some embodiments, the WAN 502 may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 503 is any computer system that is used and controlled by an end user (for example, a customer of an enterprise that operates computer 501), and may take any of the forms discussed above in connection with computer 501. EUD 503 typically receives helpful and useful data from the operations of computer 501. For example, in a hypothetical case where computer 501 is designed to provide a recommendation to an end user, this recommendation would typically be communicated from network module 515 of computer 501 through WAN 502 to EUD 503. In this way, EUD 503 can display, or otherwise present, the recommendation to an end user. In some embodiments, EUD 503 may be a client device, such as thin client, heavy client, mainframe computer, desktop computer and so on.

REMOTE SERVER 504 is any computer system that serves at least some data and/or functionality to computer 501. Remote server 504 may be controlled and used by the same entity that operates computer 501. Remote server 504 represents the machine(s) that collect and store helpful and useful data for use by other computers, such as computer 501. For example, in a hypothetical case where computer 501 is designed and programmed to provide a recommendation based on historical data, then this historical data may be provided to computer 501 from remote database 530 of remote server 504.

PUBLIC CLOUD 505 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 505 is performed by the computer hardware and/or software of cloud orchestration module 541. The computing resources provided by public cloud 505 are typically implemented by virtual computing environments that run on various computers making up the computers of host physical machine set 542, which is the universe of physical computers in and/or available to public cloud 505. The virtual computing environments (VCEs) typically take the form of virtual machines from virtual machine set 543 and/or containers from container set 544. It is understood that these VCEs may be stored as images and may be transferred among and between the various physical machine hosts, either as images or after instantiation of the VCE. Cloud orchestration module 541 manages the transfer and storage of images, deploys new instantiations of VCEs and manages active instantiations of VCE deployments. Gateway 540 is the collection of computer software, hardware, and firmware that allows public cloud 505 to communicate through WAN 502.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 506 is similar to public cloud 505, except that the computing resources are only available for use by a single enterprise. While private cloud 506 is depicted as being in communication with WAN 502, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 505 and private cloud 506 are both part of a larger hybrid cloud.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

Improvements and modifications can be made to the foregoing without departing from the scope of the present invention.

What is claimed is:

1. A computer-implemented method for generating phylogenetic inferences using a distributed computing environment with an in-memory database, the computer-implemented method comprising:
inferring phylogenetic relationships concurrently with a sequencing operation, wherein the inferring comprises, while sequencing data is in production by a sequencing machine:
accessing the sequencing data;
mapping sequence reads from the accessed sequencing data to identify sequence reads corresponding to orthologous genes of interest; and
placing the identified sequence reads into respective namespaces for the corresponding orthologous genes in the in-memory database.

2. The computer-implemented method of claim 1, wherein the inferring further comprises:
at a first interval, for each of the respective namespaces, gathering sequence reads in the namespace to create a representation format; and
generating phylogenetic inference trees for the respective namespaces.

3. The computer-implemented method of claim 2, wherein the representation format is created using a distributed multiple sequence alignment.

4. The computer-implemented method of claim 3, wherein the inferring further comprises passing the representation format to a distributed phylogenetic inference program for providing phylogenetic inference trees for the namespace.

5. The computer-implemented method of claim 3, wherein the inferring further comprises placing the representation format back into the namespace to reduce cost as new sequences are allocated to the namespace.

6. The computer-implemented method of claim 2, wherein the inferring further comprises:
at a second interval, gathering the phylogenetic inference trees from the respective namespaces; and
applying computational approaches to infer, based on the phylogenetic inference trees, a global tree that takes into account all the orthologous genes.

7. The computer-implemented method of claim 6, wherein the placing the identified sequence reads into the respective namespaces, the gathering the sequence reads, the gathering the phylogenetic inference trees, and the applying the computational approaches take place concurrently, and wherein the first intervals are more frequent than the second intervals.

8. The computer-implemented method of claim 2, further comprising providing a visualization of the phylogenetic inference trees while the sequencing data is in production.

9. The computer-implemented method of claim 1, further comprising providing a feedback signal to halt the sequencing operation when relevant phylogenetics information is available to a user.

10. The computer-implemented method of claim 1, wherein the sequence reads are mapped using short read alignment.

11. The computer-implemented method of claim 1, wherein the inferring further comprises, based on the mapping, placing non-identified sequence reads into the in-memory database without namespacing.

12. A system for generating phylogenetic inferences using a distributed computing environment with an in-memory database, comprising:
a processor set;
one or more computer-readable storage media; and
program instructions stored on the one or more computer-readable storage media to cause the processor set to perform operations comprising:
inferring phylogenetic relationships concurrently with a sequencing operation, wherein the inferring comprises, while sequencing data is in production by a sequencing machine:
accessing the sequencing data;
mapping sequence reads from the accessed sequencing data to identify sequence reads corresponding to orthologous genes of interest; and
placing the identified sequence reads into respective namespaces for the corresponding orthologous genes in the in-memory database.

13. The system of claim 12, wherein the inferring further comprises:
at a first interval, for each of the respective namespaces, gathering sequence reads in the namespace to create a representation format; and
generating phylogenetic inference trees for the respective namespaces.

14. The system of claim 13, wherein the representation format is created using a distributed multiple sequence alignment.

15. The system of claim 14, wherein the inferring further comprises passing the representation format to a distributed phylogenetic inference program for providing phylogenetic inference trees for the namespace.

16. The system of claim 14, wherein the inferring further comprises placing the representation format back into the namespace to reduce cost as new sequences are allocated to the namespace.

17. The system of claim 13, wherein the inferring further comprises:
at a second interval, gathering the phylogenetic inference trees from the respective namespaces; and
applying computational approaches to infer, based on the phylogenetic inference trees, a global tree that takes into account all the orthologous genes.

18. The system of claim 13, wherein the operations further comprise providing a visualization of the phylogenetic inference trees while the sequencing data is in production.

19. The system of claim 12, wherein the operations further comprise providing a feedback signal to halt the sequencing operation when relevant phylogenetics information is available to a user.

20. A computer program product for generating phylogenetic inferences using a distributed computing environment with an in-memory database, comprising:
one or more computer-readable storage media; and
program instructions stored on the one or more computer-readable storage media to perform operations comprising:
inferring phylogenetic relationships concurrently with a sequencing operation, wherein the inferring comprises, while sequencing data is in production by a sequencing machine:
accessing the sequencing data;
mapping sequence reads from the accessed sequencing data to identify sequence reads corresponding to orthologous genes of interest; and
placing the identified sequence reads into respective namespaces for the corresponding orthologous genes in the in-memory database.
